(19) United States
(12) Patent Application Publication
(10) Pub. No.: US 2022/0405319 A1
(43) Pub. Date: Dec. 22, 2022

(54) SYSTEM AND METHODS OF USE FOR ADAPTIVE DATA EXCHANGES ON A NETWORK WITH REMOTE MONITORING

(71) Applicant: On Track Technologies Incorporated, Durham, NC (US)

(72) Inventor: Penny Shikowitz, Durham, NC (US)

(21) Appl. No.: 17/713,213

(22) Filed: Apr. 4, 2022

Related U.S. Application Data
(60) Provisional application No. 63/170,182, filed on Apr. 2, 2021.

(51) Int. Cl. G06F 16/45; H04W 4/38; G06F 16/25

(52) U.S. Cl. CPC G06F 16/45; H04W 4/38; G06F 16/254

(57) ABSTRACT

A system provides communications between local device installations and a remote monitoring computer and includes a plurality of computerized devices communicating on a bi-directional network. At least one local device transmits local device data to a remote monitoring computer on the bi-directional communications network. An interface computer receives the local device data and formats the local device data for further transmission to the remote monitoring computer, wherein the interface computer has at least one processor and computer memory storing interface software that implements a method of translating data of any kind into a preferred format. The method includes storing the local device data in the computer memory of the interface computer; transforming the local device data into a formatted data set; and transmitting the formatted data set to at least one remote monitoring computer.

### CROSS-REFERENCE TO RELATED APPLICATION

This application is a non-provisional filing claiming priority to U.S. Provisional Application Ser. No. 63/170,182 filed on Apr. 2, 2021, which is incorporated by reference as if set forth in its entirety below.

### STATEMENT REGARDING FEDERALLY FUNDED RESEARCH

None.

### FIELD

This application is generally related to a network of sensors and other local devices connected to a bi-directional network that are monitored remotely by computer systems that utilize data exchange protocols that can be selectively utilized to adapt the data for more uses and monitoring applications.

### BACKGROUND

The term Internet of Things (IoT) has been used for over 20 years, and although it sounds complicated and cloaked in a bit of mystery, it is simply a network of internet connected objects able to connect and exchange data. The connected “things” have sensors and that provides numerous opportunities.

Data collection is one of the main operations performed with these sensor networks. The variety of sensors with dedicated measurement modalities has broadened the capabilities and applications across all industries. Use of technologically advanced sensors with connectivity has enabled increased insights, facilitating better data-based decisions, processes, and outcomes.

Multiple sensors are found in nearly all devices, each with specific measurement capabilities generating unprecedented amounts of data. An immense amount of data is being generated at an exponential rate; however, the question remains, is there a way to receive, assimilate, and use the data for better performance solutions?

The present and future of civilization are impacted by sensor technology, and although this advancement not only brings incredible opportunities, it also presents significant challenges. Sensor technology is complex in both configuration and the abundance of data generated. The delivery of data and assimilation of the data into a usable and understandable solution presents challenges for most companies. It is exciting to imagine having access to real-time data of any configuration because knowledge of operational data is critical. That excitement quickly fades when faced with the volume of data that is generated without the ability to make it immediately actionable.

The world has forever changed with the prevalent integration of sensors into a vast range of products used to measure various specifics of data. Sensors represent technologies on which effective situational awareness strategies are built with the capacity to perceive, analyze, and act upon a situational status within a preset context. Although the phrase situational awareness has traditionally been used within the military in relation to tactical decision-making, it is now appropriately applied to advanced technologies and operational processes.

Sensor technologies include connectivity, analytics, and cloud server approaches that expand the impact of data on an organization's performance. To successfully integrate the data, it must be transformed for delivery into the current system with information that is actionable in real-time. This requires highly complex capabilities and experience in delivering data into disparate systems. Whether an industrial manufacturer is expanding diagnostic and performance capabilities, or a medical practice is seeking to provide remote medical monitoring, the possibilities of sensor solutions are nearly infinite.

Sensors measure defined criteria, requiring a data delivery system to various applications and then actuating a response of situational status. The information must be bi-directional, able to take measurements, deliver the data into existing systems, and when needed trigger an action response.

A connected solution includes four components: sensors, network, storage, and analytics. Anything that is attached to a sensor has the potential to benefit from vast stores of data production, often with the connection of an infinite number of sensors. The network provides the vehicle to share the collected data. Data storage is possible in on-premises solutions however, the sheer amount of data being relayed has made cloud-based storage the preferred option while allowing the data to be accessible. The fourth component, analytics, relates to the exponential increase of information generated, requiring sophisticated processors, and analytics software to assimilate the data into usable formats.

An example of sensor applications on a device is the mobile phone. For example, a mobile phone includes accelerometer sensors that are used to measure velocity but also can help determine the orientation of the phone as it rotates, allowing the screen to rotate appropriately as detected by the situational awareness. Within this same phone are light sensors which transform the light into pixels, creating the images captured by either still or video photography. Another sensor delivers object identification messaging to the camera within the photography mode to enhance the image color and quality, or possibly also used as the locking mechanism.

Sensors, therefore, can measure temperature, proximity, acceleration, pressure, light, sound, touch, color, humidity, tilt, flow and levels, chemicals, and biometrics, helping companies transform concepts into smart, connected solutions. Sensors are a necessary and important component of situational awareness technologies; however, it is the compilation of the devices, real-time computing, geographic information, generated data, and delivery into a system that realizes the technological value.

Devices equipped with sensors performing various functions can give an organization a broader view of how a product is functioning in real-time, creating a path to improving performance, mitigating potential failures, and more. When using multiple sensors on one device, data increases exponentially requiring specific processes for ingesting, storing, processing, and analyzing the data.

Companies often decide to begin using the newest technologies available, understanding that gathering data is important to performance decision-making and other critical analyses. Researchers do not go far into the process before deciding it is too expensive with profound limitations. A major computer and data processing company recently polled 3,000 decision makers regarding the adoption of such technologies only to conclude that 30 percent of projects failed in the proof-of-concept stage due to implementation that seemed too expensive for the company to realize bottom-line benefits.

Companies, therefore, will benefit from advanced technologies and the gathering of useful data, but only if the integrity of raw data can be maintained while pulling criteria-based subsets of that data for delivery into existing systems. The resources required to generate and capture the data is wasted if it cannot be meaningfully consumed.

The ability to realize the value of data being generated requires more than merely putting sensors on objects and pointing the data to a receiver. Organizations investing in purely connecting objects and gathering data without implementing technologies that assist in the analysis and assimilation of that data are creating an information flood that ultimately will be unusable. Too often organizations do not have a system to receive, analyze, and actuate the expansive amount of data.

Most organizations simply do not have the tools to pull value from the data, at which point it is an unrealized asset. Organizations need solutions to automate and combine various types of information from multiple sources, then analyze, and implement the results into current processes. The first stage of data captured by sensing devices provides information that is raw or unprocessed combined with statistical noise, requiring the data to be parsed. There must be protocols to extract the necessary measurements, and semantics without losing the integrity of the data as a whole.

Companies will generally “normalize” data so that it meets preset parameters prior to sending the data to their cloud. Once normalized, the data becomes a subset of the original thus losing overall integrity, and future analytical opportunities. Organizations normalize data in an effort to manage the massive amount of data generated while realizing the problematic nature of losing the original information. The decision is based on necessity of data management rather than creating optimal conditions for future analytics. Without the capability of varied data extraction at any time, there can be no basis for gained information knowledge.

In addition to the concern of maintaining original data information, organizations face the investment of time and resources to implement a new system, processes, and staff to monitor and facilitate the data into use. The challenges require solutions that allow for sensor generated data to be captured, relayed with no limitations into various receiving endpoints without loss of information for future use. The solution would require the ability to deliver data with varied presets of criteria to applicable receivers, ideally delivered into existing systems without custom configuration requirements at the receiver end of the information chain.

In the competitive landscape of companies across all industries, they require a solution for managing data that few organizations currently have. For the successful management of sensor generated information it is critical for organizations to adopt data relay technologies that are adaptable, actionable, and easily assimilated into their current systems for the data to provide real value.

One challenge lies in increasing data security by delivery of filtered relevant data to specific endpoints. Sensors attached to devices are taking measurements and creating the data used in various analysis, and decision-making endeavors. While an organization may want data from a specific sensor to be delivered to a specific receiver, they may not want all the data to be shared. An organizations' data is generally linked to proprietary assets and while there is a desire to share some information with a component supplier, it is not advantageous to allow the sharing of all of the generated data.

With the explosion of the use of sensors and expansive connectivity options it becomes increasingly more important to adopt cutting-edge technologies that allow for necessary customization through filters, notifications, and alerting. To guard against increased security threats, organizations require multilayered security strategies that allow for intelligent data that is actionable, scalable, and sustained without disrupting service. The data delivery solution must be secure and private with built in protocols for security. One way to maintain security of the information is to assign delivery of various data components to varied endpoints, thus protecting the entire data information by not allowing every recipient to receive all information.

One of the most exciting aspects of technology is the consistency of its ever-changing applications to common life experiences. An evolving approach to health care management has accelerated the widespread implementation of telehealth, and applications of remote patient monitoring (RPM). Telehealth is defined as the distribution of health-related services and information via telecommunication technologies, while RPM technology enables monitoring of patients outside of conventional clinical settings such as in the home or in a remote area. The outbreak of Covid-19 increased awareness around the complexities of providing health care while at the same time protecting the community and health care providers alike. The widely accepted adoption of telehealth is strengthened with remote monitoring technologies providing the devices are easy to operate by the user with seamless integration of data by clinicians.

Similarly, Remote Medical Monitoring (RMM) is the technology of data collected by a device and relayed to healthcare providers. Devices with RMM technology can remotely monitor patient vitals and other symptoms used as indicators of changes in medical condition for better patient management. The technology is approved for medical cost reimbursements led by government initiatives, resulting in a shift to value-based care, and a greater continuum of care. In addition to actionable patient care, RMM is poised to play a significant role in protecting the health of densely populated conditions such as daycare, schools, correctional facilities, and workplaces. The combined use of telehealth and RMM will become the new norm of health management and preemptive health protection. Benefits will be realized by patients, physicians, and insurance providers with better patient outcomes, and reduced costs of medical care.

Telehealth, RPM and RMM have positive impacts on health care costs, and the financial stability of providers. Adapting to changes of health care and infectious disease management will require the use of Health Insurance Portability and Accountability Act of 1996 (“HIPAA”) compliant technologies that are easily implemented within the population, and seamlessly integrated into electronic health record systems (EHRs), electronic medical record systems (EMRs), and medical software systems. Adaptable, flexible, and reliable, secure data exchange solutions are necessary now, more than ever.

Medical care for people in remote locations is especially challenging for patients with progressive diseases in specialty healthcare practice areas such as nephrology, cardiology, and pulmonology. Remote locations are generally defined as rural communities but also include patients traveling domestically and abroad, further complicating the monitoring of patients with chronic health conditions. Some patients experience mobility challenges while others have conditions negatively impacted with the stress of movement from home to clinic or hospital, for scheduled monitoring.

The overburdening of medical resources in times of health crisis further impacts access to medical care. Healthcare workers have tremendous demands on their time, further complicated with the tasks required to retrieve and record biometric data such as blood pressure, heart rate, blood oxygen saturation, weight, blood glucose, and temperature.

For instance, patients with chronic illnesses require constant medical monitoring as a valuable component in identifying real-time changes in condition, requiring treatment for slowing the progression of the disease. Various factors often impede the ability for patients to access the care needed such as overburdened and unavailable medical resources, costs, and the progression of illness creating mobility difficulties.

During times of communicable infectious disease outbreaks, densely populated conditions exacerbate the situation to critical levels if health conditions are not monitored within the population. This situation is applicable to child and adult care facilities, schools and universities, correctional facilities, work environments, and military bases. Industries are also at risk of viral spread, potentially resulting in workplace shutdowns, and food recalls, risking the health of employees, company revenues, and the general population. Historical evidence proves the rampant spread of viruses in situations of close interaction with further spread to friends and family members.

The rise of health care costs has resulted in a movement from uncoordinated fee-for-service delivery of health services to a coordinated, accountable, and patient centric fee for value-based care. The ability for medical facilities to thrive will depend on achieving high-quality care thresholds resulting in better patient outcomes, at lower costs. To realize these goals, it is imperative for providers to utilize traditional hands-on care, telehealth for accessibility, and RMM for patients requiring an increased level of medical attention. The exorbitant costs of operating a physicians' clinic or hospital require the adoption of technologies that allow for an increased number of patients with less in person visits and reduced costs to patients without sacrificing the quality of care.

A common scenario would include a patient being discharged after a successful surgery, only to be readmitted within 30 days. This sequence occurs with a range of 10 to 20 percent of all surgeries, as charted by the renowned Mayo Clinic. Readmissions are frustrating to patients and physicians, while also costing the industry billions of dollars, annually. Previous patient care has been heavily skewed towards hospital and in-person medical care; however, there exists a trend and need for wide-spread implementation of telehealth, the use of remote medical devices, remote patient monitoring (movement, ambient conditions, physiological data monitoring, and the like, when applicable to realize a shift in the quality of medical care.

The most important concerns for patient adoption of remoted medical monitoring are uncomplicated use, comfort, reliability of the relayed data, and patient training in the proper device usage and accountability for use consistency. If the patient loses confidence in the reliability of the data being delivered to the monitoring clinician, the likelihood of continued use is greatly reduced. RMM with a reliable data delivery platform that is HIPAA compliant is crucial in maintaining patient confidence and participation.

RMM provides technology-driven opportunities for better health management. Physicians must have the flexibility to choose appropriate devices to fit their patients' needs, combined with medical monitoring capabilities enabled through adaptable data exchange. The required solution must go beyond medical monitoring and alerts when measurements exceed acceptable thresholds, but must also include the capability to alert during instances of patient noncompliance in taking biometric measurements. Notifications and alerts are optimal when linked to calendaring with any increment of time as defined by the clinician. For optimal outcomes the data must be granular, ingested without limitations, adapted to the specific data presents, with high frequency delivery directly into the physician's platform of choice.

Network availability has been a consistent challenge with RMM as not all patients have a home network, however, with the prevalence of mobile phones, connectivity has become more prevalent. The landscape of patient care opportunities has resulted in better patient outcomes through the use of medical devices for optimizing data management, and high frequency delivery of the information to healthcare providers.

Applications for RMM optimized with a data delivery system provides an emerging technology that's capable of delivering actionable data in real-time from integrated medical devices. Most solutions normalize health data and store the data in the cloud rather than sending the granular data in real-time to the RPM application and monitoring clinician. Delivery of various data presets to multiple receivers such as a team of attending physicians is necessary for enhanced, comprehensive patient management.

Clinics and healthcare facilities often operate with legacy systems, or multi tiers of different operating systems and platforms. It is not uncommon to find a medical facility with multiple applications and countless departmental solutions. Acceleration in RMM adoption can only occur when the data relay application is easily integrated, adaptable, and comprehensive, further proving the necessity of an adaptive data exchange solution.

Providing RMM to a large number of patients generates unprecedented amounts of data to be ingested and shared. When a healthcare facility operates with outdated software and hardware technology, operational inefficiencies are inherent causing catastrophic decision-making and treatment delays, increased costs, and overlaps in duplicating research and other tasks. A solution is required for the seamless transmission of actionable data to multiple endpoints (example: varied medical specialists, insurance providers, primary physician), with a secure, cloud agnostic exchange technology.

Alerts are linked to data presets with notifications sent when a metric has crossed the criteria threshold. Optimal efficiencies allow for monitoring processes to deliver timed notifications and data updates. With push notifications, clinicians gain valuable insights into any variable of defined medical monitoring. Alerting and timed notifications are critical components for successful utilization of real-time medical monitoring data. Physicians face challenges in finding solutions that provide remote monitoring of patients without creating alert fatigue caused by continual notifications before the patients' measurements cross acceptable thresholds. A solution must be adaptable in defining varied data presets, actionable with bi-directional alert notifications, and flexible in the delivery of information to multiple receivers.

RMM, therefore, offers technology solutions with far reaching benefits in patient management resulting in a higher level of patient engagement when used appropriately. Telehealth combined with RMM technologies provide the structure necessary for remote patient care with the ability to mitigate potential declines in health as foreseen by real-time data delivery. The technology increases the healthcare of people residing in remote areas, travelers, those isolated in the presence of widespread virus outbreaks, people with limited mobility, and the population at large.

Telehealth combined with RMM increases the opportunity for better clinical outcomes with many medical specialties including the complex care of nephrology patients. Remote patient management offers renal health care providers and patients with end-stage kidney disease opportunities to embrace home dialysis therapies with greater confidence and the potential to obtain better clinical outcomes. Remote patient management has exciting potential to improve home dialysis patient care and home modalities uptake, to improve quality of life, and to reduce cost.

RMM has proven to be a critical key to reducing readmission rates while making health care cost-effective and accessible for persons with chronic illness, those in post-operative recovery, and patients undergoing extensive physical therapy.

There are, however, still challenges present in RMM data delivery and use applications. Medical practices must manage a large-number of patients per clinic, while providing value-based care, with tight levels of staffing. With this optimal criteria, traditional solutions are not sufficient. The use of RMM is required for medical professionals to meet increased patient demands with a validated data-based knowledge of each patient. Successful RMM requires integration of remote medical devices configured with advanced data exchange capabilities to ingest large amounts of data and deliver varied presets of information to multiple endpoints, from a single source. Clinicians must be able to select the device best suited for patient use with confidence of reliable and accurate high frequency delivery of information. Without technically advanced applications, physicians will lose valuable time in recognizing medical condition trends or other critical situations with potentially harmful repercussions.

The U.S. has a rapidly aging population at a time of decreased numbers and shortages of healthcare professionals. RMM addresses these challenges by providing the ability to better manage patients with chronic illnesses that are inherent with aging while maintaining an optimized level of care. While the preferred application of RMM is accomplished with Bluetooth enabled medical devices in order to eliminate human errors in recording data, manual input must also be supported via RMM.

### SUMMARY

A system provides communications between local device installations and a remote monitoring computer and includes a plurality of computerized devices communicating on a bi-directional network. At least one local device transmits local device data to a remote monitoring computer on the bi-directional communications network. An interface computer receives the local device data and formats the local device data for further transmission to the remote monitoring computer, wherein the interface computer has at least one processor and computer memory storing interface software that implements a method of translating data of any kind into a preferred format. The method includes storing the local device data in the computer memory of the interface computer; transforming the local device data into a formatted data set; and transmitting the formatted data set to at least one remote monitoring computer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Reference will now be made to the accompanying drawings, which are not necessarily drawn to scale.

FIG. 1 is a schematic of a computer environment used in accordance with the disclosure herein with particular reference to medical uses of the disclosed technology.

FIG. 2 is a schematic of a computer environment used in accordance with the disclosure herein with a plurality of sensor types providing any data type a local computer for further transmission to other systems on a bi-directional computer network.

FIG. 3 is a schematic of a kind of sensor data transmitted by a local computer on a network for further processing by a hub application hosted by a computer on the network, an adaptive data exchange computer, and ultimately by a remote monitoring center according to the methods and apparatuses of this disclosure.

FIG. 4A is an example display of data converted by the hub application from binary format of FIG. 3 to a second format for use by an adaptive data exchange, such as JSON in FIG. 4, including human readable information, meta data, timestamps, and the like.

FIGS. 4B and 4C are example data converted by an adaptive data exchange into a preferred protocol for use by a remote monitoring computer.

FIG. 5 is a flow chart of one method of using the technology of this disclosure in terms of data delivery as a service discussed in this disclosure.

FIG. 6 is a flow chart illustrating one example use of the technology of this disclosure used in the context of an Alzheimer's Geospatial System (AGS).

FIG. 7 is an example flow chart illustrating that the technology of this disclosure is data agnostic and can be formatted for use with a plurality of different monitoring centers and intermediate computers.

FIG. 8 is a schematic illustration of certain information relay capabilities of the data delivery and data monitoring technology of this disclosure.

FIG. 9 is an example flow chart of using the disclosure herein in the realm of medical monitoring, particularly for elder care and Alzheimer's Disease.

FIGS. 10A and 10B are example sets of screen shots for one embodiment of this disclosure that is used in remote medical monitoring in the context of Alzheimer's geo-fencing options.

FIG. 11 is a schematic illustration of a computerized environment in which more than one network participant shares data and information according to embodiments of this disclosure.

FIG. 12 is a schematic illustration of a computerized environment in which more than one third party (asset manager) can simultaneously access data for multiple network participant data partitions.

FIG. 13 is a schematic illustration of an example electronic control unit and associated computer hardware used in conjunction with the systems and methods of this disclosure.

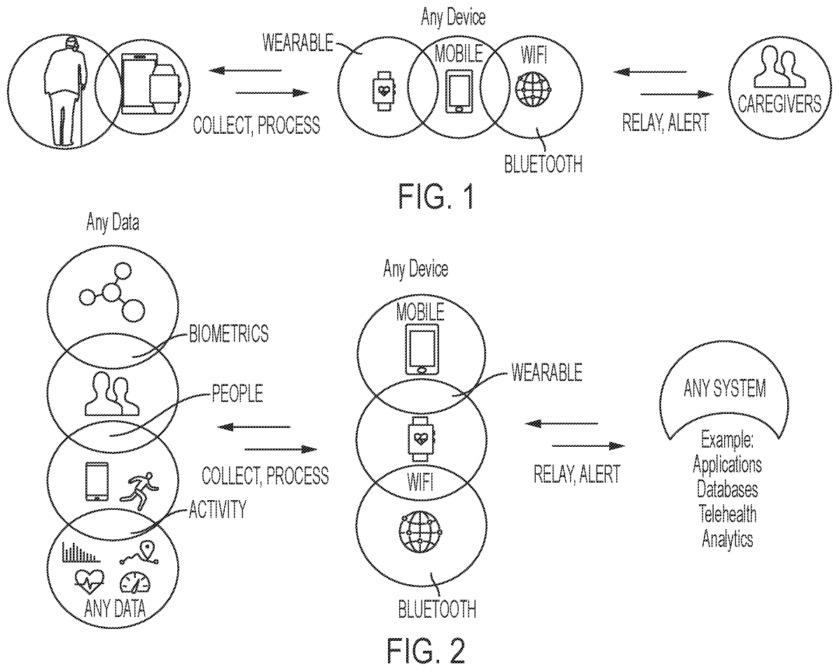

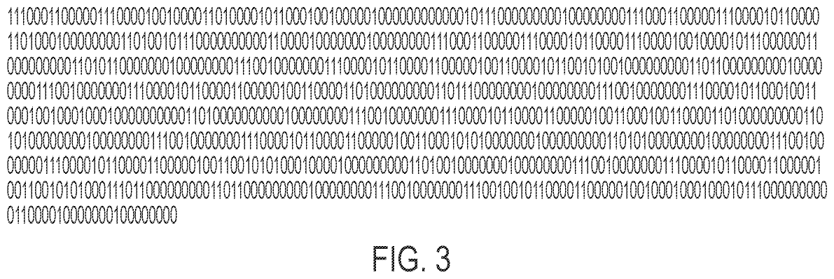

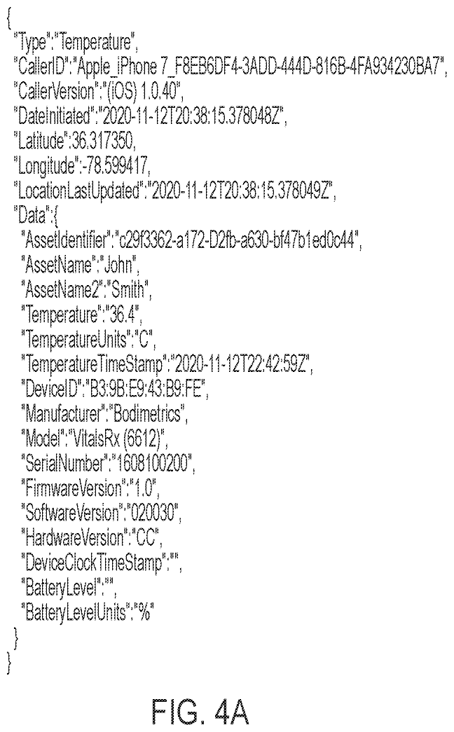

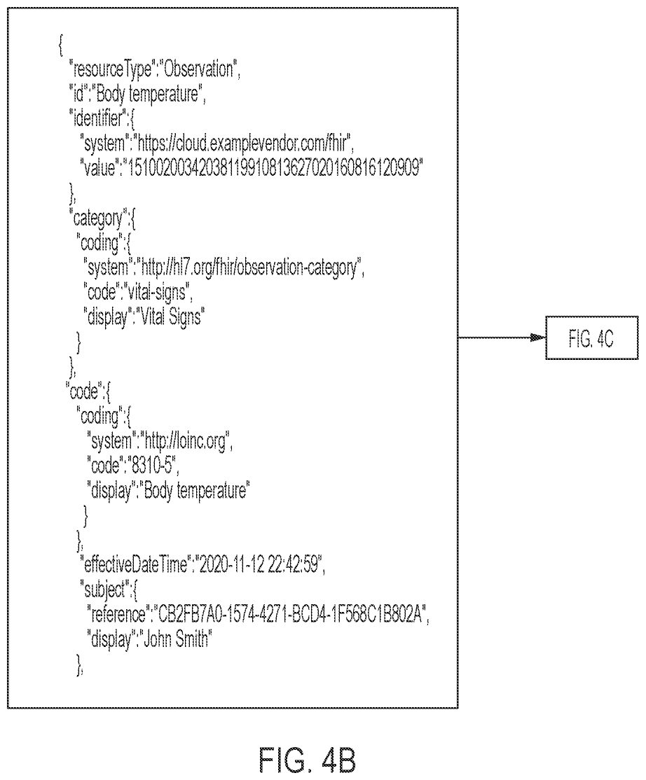

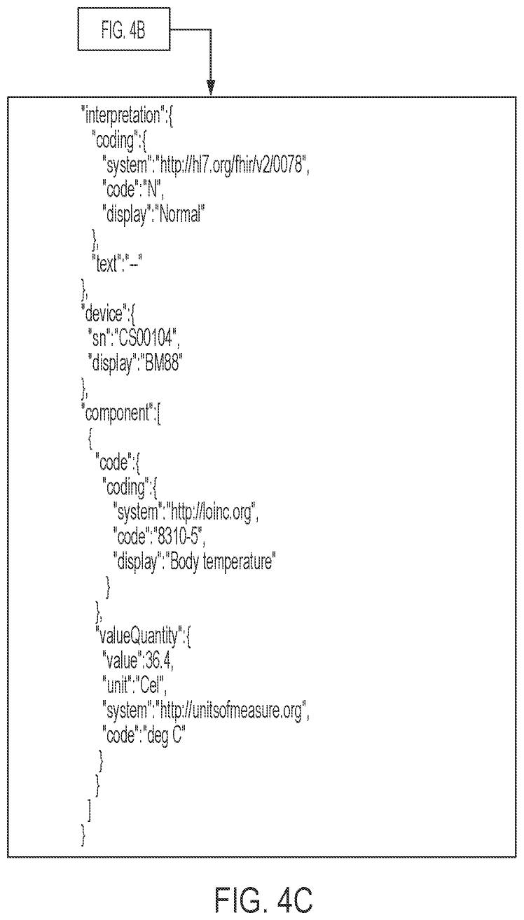

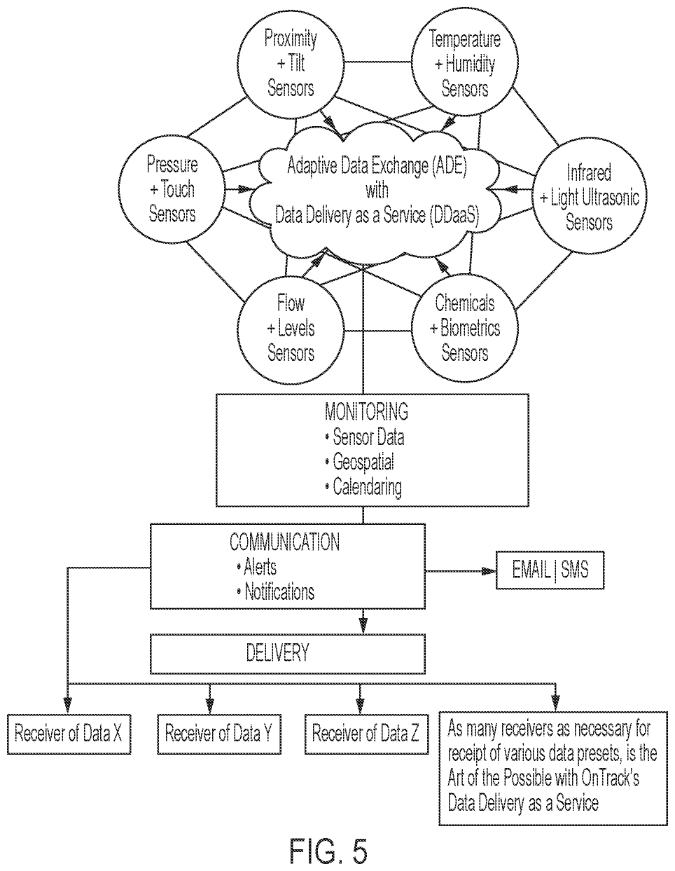

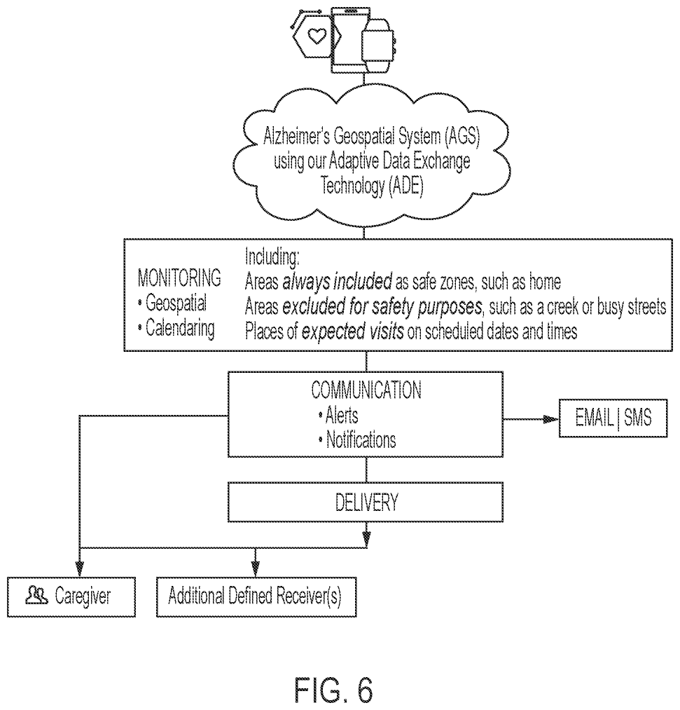

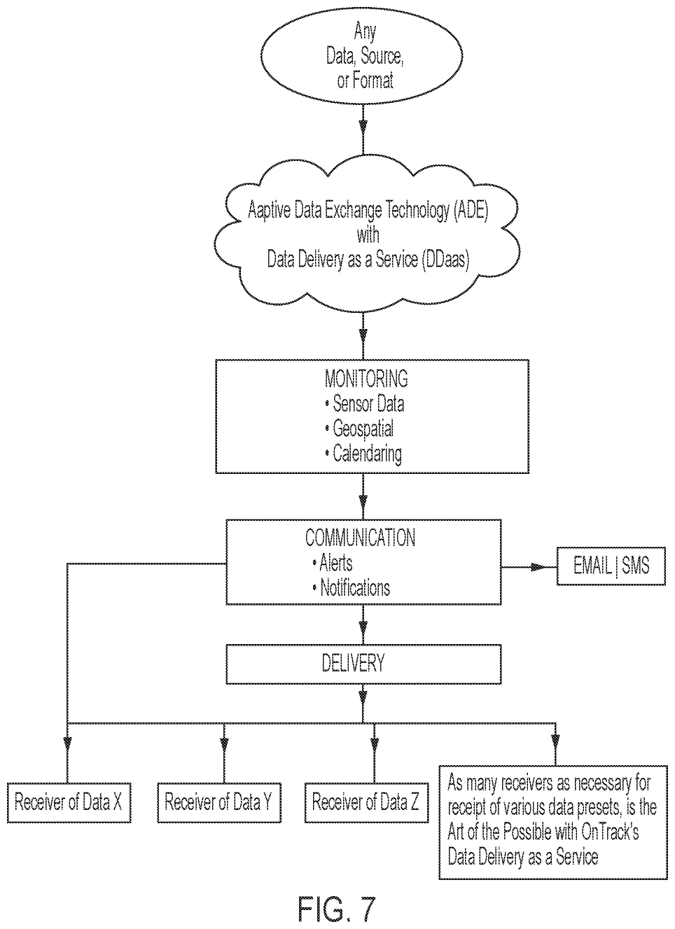

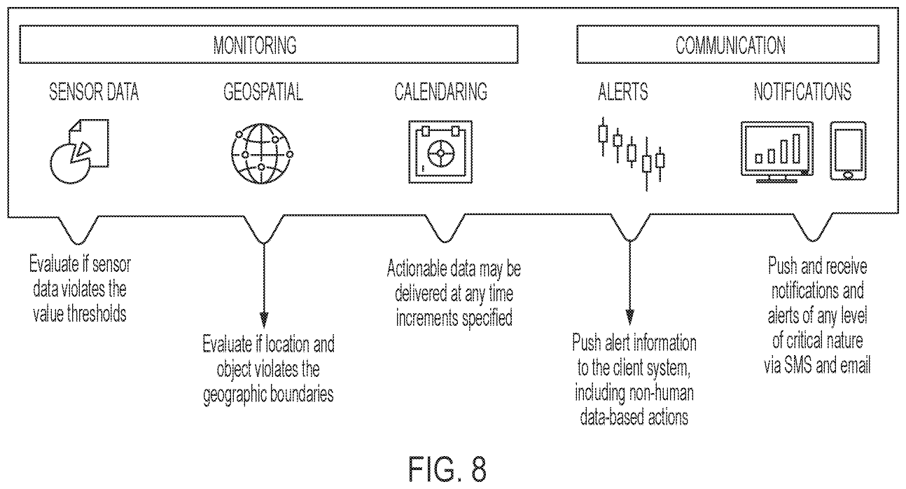

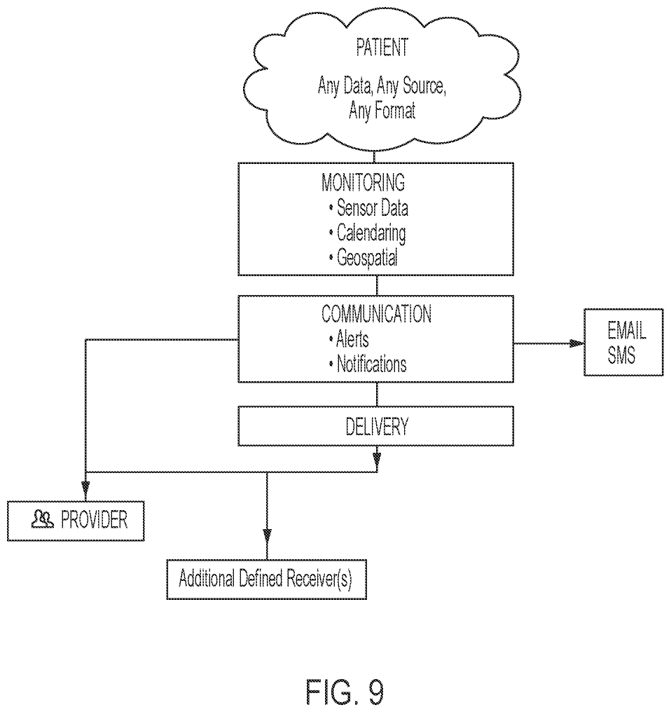

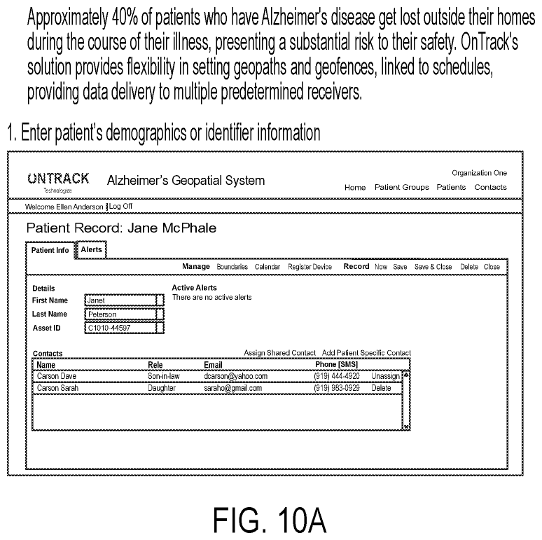

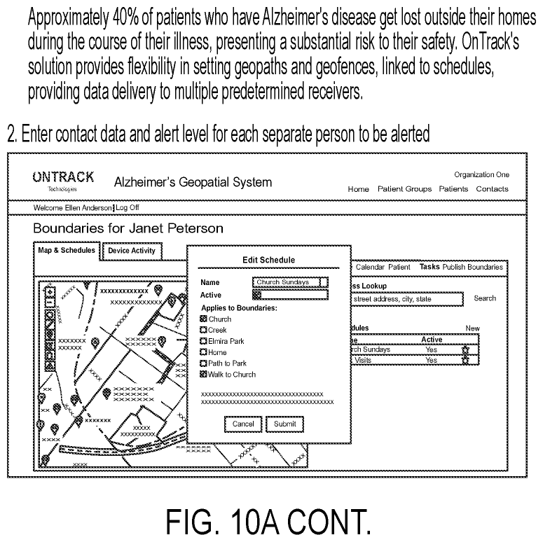

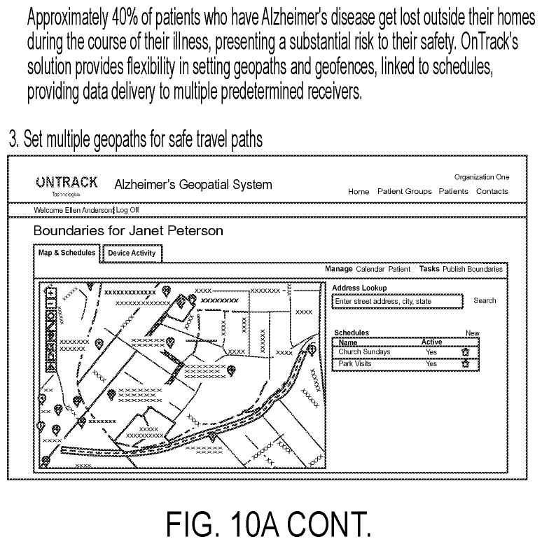

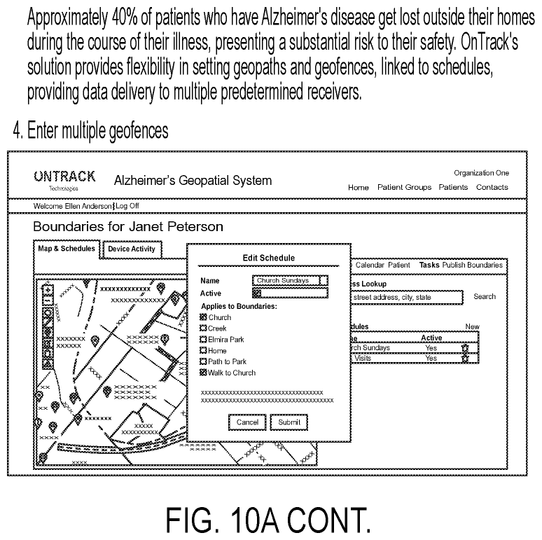

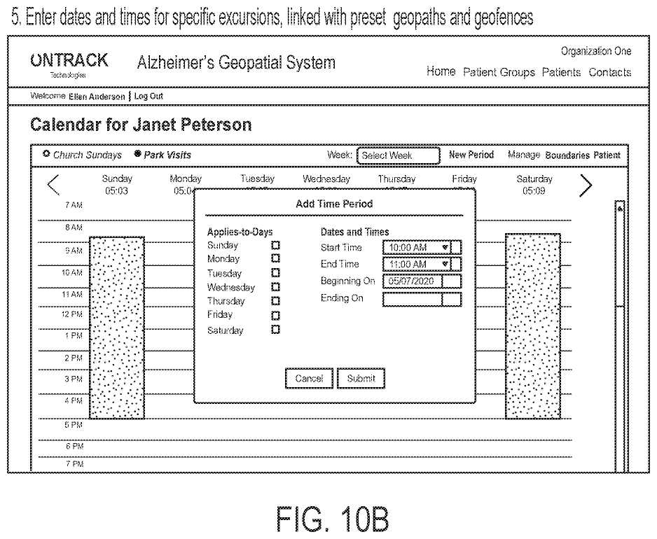

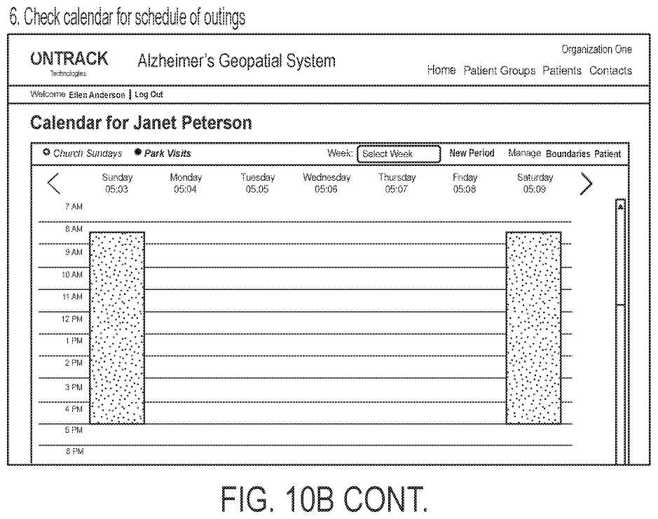

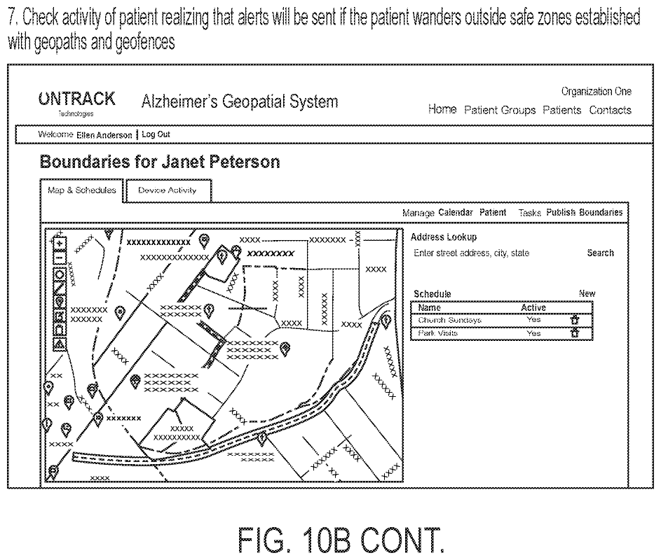

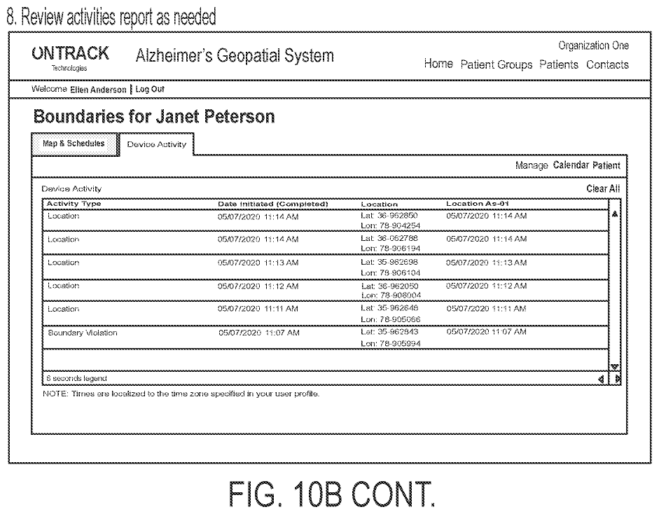

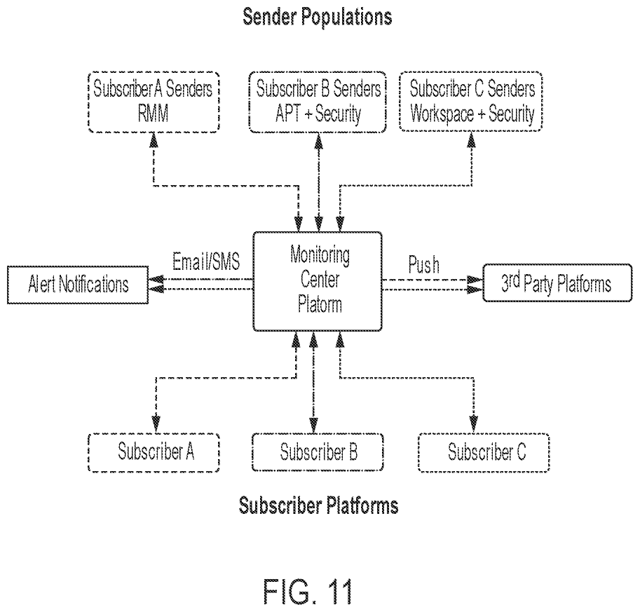

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

In some embodiments, the technology presented herein includes retrieving data measurements from one sensor and delivering predefined aspects of the data to one receiver, and different measurements of the same information to another receiver, without loss or corruption of the original raw data. This would be a solution to the problem of oversharing data, while allowing multiple endpoints to receive only the data that is relevant to their specific role and task. Organizations, therefore, can use this technology to enable the sensor collected data to be pulled from the cloud and delivered with various preset filters, into multiple predefined receiver points. Consider this to be a necessary type of data “multi-tasking” to address not only the complexities of generating massive amounts of data without implemented technologies to deliver it in a way that optimizes the application and use as well as the complications of filtering data based on the situational awareness and situational status.

In the quickly changing environment of commerce, research, and technology, raw data capture and protection must be managed while also allowing for filtered data to be pulled from the cloud and delivered to various receivers. The identification of needed capabilities include filtering the data based on criteria thresholds, calendaring with date, time, or any increment of time, and notifications and alerts when the data measurements are outside the preset thresholds. The system must be able to self-monitor for its environment and make necessary adjustments based on the situational status.

The optimal functionality of the technology disclosed herein includes the capture of all raw data generated while pulling information from the cloud that pertains to the specific presets and filters, without losing the raw data as a whole. Also important is the ability to filter various aspects of the same raw data for delivery to multiple receivers, each needing various subsets of that data. It is only with this defined flexible delivery capability that the data is optimally useful without normalizing and losing original data most probably required at some future point in time.

One concept discussed herein is the Adaptive Data Exchange (ADE), which includes computer hardware and software implementing the following features:

Intelligent: Data is actionable, manageable and useful.
Bi-directional: Push and pull data between disparate systems, devices, applications, and databases.
Agnostic: Virtually any use case, application, data type, and transmission mode.
Flexible: Rapidly customized for any use case.
Scalable: Scaled and sustained without disrupting service.
RESTful API: Easily integrated into existing applications and workflows.
Proven: Cloud agnostic ADE technology can be adopted for use by government and military agencies.
Secure & Private: Built-in protocols for data security.
Cloud Agnostic: Provides cloud-based access to remote monitoring data.

The Adaptive Data Exchange supports multiple use cases. It communicates efficiently with customer applications, remote devices, and third party systems within an infinite range of industries. The ADE may be multi-tenant with the capability of delivering predefined modalities to various data systems. It includes options for rapid integration and deployment, validates and ingests data from devices and applications, and then deploys to predefined systems. The solutions provided herein include incremental deployment for high availability and data resilience.

In the embodiments disclosed herein, the ADE may include real-time updating options with presets that are determined for data relay at real-time. There are options for treating different data with different protocols on a granular level (i.e., providing 100% of data collected in a configuration that is consistent with needs at the receiving party). In other situations, the embodiments of this disclosure may include condition monitoring (i.e., monitoring specified parameters, such as vibration, temperature, or any defined conditions, and relaying the data to various endpoints).

In other embodiments, the ADE provides even more options such as the following:

Rules Based Alerting: Delivers customized commands and configurations with real-time alerting.
Pushes: Receive, validate, and queue payloads in highly configurable, generic data ingest endpoint.
Asset Verification: Confirm payload's specified asset exists for subscriber; auto-retrieve from subscriber if not.
Subscriber Requests: Validate and queue commands & settings sent by subscriber platform for retrieval by local device.
Assets: Get assets linked to the sender device/app, or find asset by name/identifier.
Videoconference: Initialize conference between sender and subscriber platform.
Settings: provide sender with default settings and white-label info for the subscriber.
Configure: Look up predefined configuration for inbound payload type.
Transform: Convert data in inbound payload to syntax, units, etc. required by each target platform configured for the asset.
Authenticate: Make secure connection to target platform.
Push: Deliver transformed payload to target platform; verify receipt and retry on failure.
Assets: Manage assets monitored by the monitoring center platform (MCP).
Senders: Manage instances of senders (e.g., RMM Hub, workplace monitoring node).
Asset-Sender Relationships: Optionally tie assets to senders in many-to-many relationships.
Alerts: Manage alert definitions and notification contacts on per-asset basis.
Subscribers: Manage collection subscribers (e.g., monitoring center platform reseller, or large organization needing internal data partitioning).
Configure: Look up user-defined boundary conditions and notification contacts.
Compare: Test data in inbound payload for values that violate boundary conditions.
Push: Deliver payload of alert details when boundary violation occurs.
Repeat/Escalate: Continue evaluating condition on user-defined periodicity and escalation settings; send repeat alerts where needed.
Notify: Send SMS text and email notifications to designated contacts; repeat on failures.
Sender device (or app) obtains measurement or receives user input for customer platform to receive. Examples include sensor reading, user-invoked command or confirmation, device status.

The claims following this disclosure also incorporate disclosure of the remote monitoring computer, referred to also as the Adaptive Data Exchange and/or the monitoring center platform. The features of the monitoring center platform (MCP) include:

MCP validates payload based on predefined configuration (use-case agnostic).
Sender device or remotely activated computer application sends payload (e.g., measurement) to an MCP endpoint.
Payload saved to processing queue in database.
MCP evaluates whether payload contains actionable information based on customer's predefined boundary condition(s), if any. If so, MCP pushes alert information to endpoint on customer platform.
MCP sends email and/or SMS text notifications to pre-designated recipients for the asset and defined alert condition.
MCP uses configurable transform functions to convert the payload to target platform specifications, then pushes the converted payload to customer and/or third-party platforms.
Customer (Subscriber) user invokes function on their platform that sends “request” payload to the MCP. Examples include a command (e.g., turn phone light on); device configuration (e.g., geographic boundary).
MCP receives the payload.
MCP validates payload based on predefined configuration (use-case agnostic).
Payload saved to Sender's request queue in database.
The Sender checks the MCP for any pending requests.
Pending requests are pulled by the sender and processed, with confirmation of receipt sent back to the MCP.

In some embodiments, this disclosure utilizes the above noted ADE to address limitations that are present in remote medical monitoring, noted above as RMM. RMM can prevent unnecessary hospitalizations and reduce readmissions, healthcare costs, and overburdening of medical facilities. The delivery of health-related information must be flexible and responsive for optimal analysis and medical treatment decisions. Most current systems are not able to ingest data without being authenticated in the format of the receiver. The technology of this disclosure provides an emerging technology with the capability to ingest data and deliver granular data as a service. The solutions presented herein are cost effective, HIPAA compliant, subscription-based RMM system with security functions allowing controlled access for data to be delivered to multiple endpoints.

Specifically, RMM provides the following in a data exchange solution:

Intelligent data that is actionable, manageable, and useful
Bi-directional delivery of data into existing platforms, with the ability to push and pull data between disparate systems, devices, applications, databases, and platforms.
System that is agnostic, applicable to virtually any use case, application, data type, and transmission mode.
Flexibility allowing for rapidly customized data delivery for any use case.
The data exchange system must be scalable, and sustained without disrupting services.
Easily integrated into existing applications and work flows.
Secure and private with built-in protocols for security.
Cloud agnostic, providing access to remote data monitoring.

As described in the claims that follow, the technology of this disclosure may be described as Data Delivery as a Service (DDaaS) with Adaptive Data Exchange (ADE) and provides a comprehensive solution for all necessary functions enabling methods and apparatuses to share data with multiple locations from a single device.

In some embodiments, the methods and apparatuses provide simplified data integration into existing platforms. To reduce the costs of data assimilation and governance challenges, medical facilities require an integrated solution of data delivery into existing software systems. ADE solutions provide DDaaS, with a user agnostic platform core that communicates with applications, remote devices, and 3rd party systems. The ADE technology validates and ingests information from various devices and applications, pushing remotely collected data with varied preset configurations and commands to multiple endpoints.

The embodiments of this disclosure further incorporate Real-Time Data Delivery and near real time data delivery. Application of near real-time alerts and actions are initiated before disruptions or medical emergencies occur. The value of data delivered in real-time is obvious, however, the delivery of the data into existing systems allowing for immediate analysis is invaluable. Simplicity, speed, security, and data accuracy are critical factors in making timely decisions, ahead of the curve.

The advantages of real-time data delivery are at the heart of successful decision-making, providing better performance outcomes regardless of the industry or use. Real-time data is invaluable as it reduces the time it takes to get information from the patient to their medical team. The information ensures the accuracy and expedience of situational awareness, including the comparison of current and historical data.

The disclosure further incorporates Alerting and Timed Notifications. Alerting and timed notifications are critical in RMM and the process of data exchange. The use of an ADE solution offers built-in, configurable functions to inform clinicians when actionable data has been received from medical device sensors linked to patients, as well as when there are absences of expected data from those sensors. For example; if a physician sets an alert temperature threshold to 100.4 degrees Fahrenheit and assigns key monitoring staff to be notified when this threshold is exceeded, the medical device sensor sends a temperature reading of 100.4+ degrees Fahrenheit, which causes the ADE to implement the high-temperature alert protocol for that sensor. Additionally, the same medical device sensor may be configured to perform one temperature reading every 2 hours, thus if the sensor fails to detect a reading after 2 hours have elapsed since the previous reading, the ADE implements a no-reading alert notification.

A poignant value of the ADE system is with the high degree of customization capabilities that can be applied per medical device including alert repeat and escalation intervals, as unintended alerts will escalate in priority until canceled. In reference to customization, notification contacts can be set at specified alert levels as well as the ability to apply do-not-disturb time periods. By implementing RMM with DDaaS, both patient and physician experience confidence in the ability to receive notifications and alerts, of any level of critical nature, providing the opportunity for immediate reactions to those notifications as required.

The solutions offered herein provide unprecedented data management capabilities enabling the physician to decide where, when, and in what format the data is to be delivered. The solution captures 100 percent of the data generated, while allowing the physician to create alerts and notifications on subsets of the data, offering the flexibility to meet any unique criteria. Most data delivery options have “if” included in the capabilities statement however, the embodiments herein may be better descripted with the word “any”:

Any actionable data may be delivered anywhere, at any time specified, and in any format that is required at any source.
The solution captures and delivers 100 percent of the data generated.
Physicians may set alerts and notifications on any subset of the data.
The solution includes calendaring, which allows for any presets of measurement and notification frequency.
The solution is bi-directional, offering necessary flexibility and functionality.

There are significant benefits in implementing RMM with DDaaS. Data sent from patients to healthcare professionals at real-time is highly important for people with chronic illnesses. RMM capabilities improve the quality of life in patients by giving them the confidence of professional monitoring and reducing inherent fears of the unknown status of illness progression. The disruption of normal, daily routines by chronically ill patients are eliminated with RMM, providing more time to be shared with their families and less time in clinics or hospitals. Patients not only realize a better quality of life without constant focus on illness, they also enjoy the benefits of better healthcare at a lower cost. RMM offers what every patient desires; increased quality of health care, less in-person medical visits, cost savings, and peace of mind.

In some embodiments, this disclosure provides flexibility of device and RMM Data Presets and Delivery. Integration may be made with any medical device chosen by a physician through cooperative effort of the device manufacturer. Within every medical facility there exists a unique structure of data requirements, software systems, and consistently evolving changes in the size, scope, and scale of information needed for decision-making analysis. The presented solutions can deliver data presets irrespective of its source, form, or structure, enabling customers to perform real-time analytics capabilities including Geospatial, Calendaring, Monitoring, Alerting, and Notifications. Most importantly with the solutions of this disclosure, biometric data is scalable with an unprecedented number of variables, and efficiently exchanges information with varied data presets between multiple physicians, clinics, and medical insurance processors, from a single originating source.

### Example Embodiment

In one example embodiment, this disclosure considers the life changing impact of using devices with sensor technology in Alzheimer's patient management. This disclosure presents an Alzheimer's Geospatial System (AGS) used with devices for measuring proximity as related to location with geopaths and geofences linked with date and time calendaring. The capabilities also identify tilt to detect and alert in the event of a fall, temperature, and humidity in relation to environmental conditions, and biometrics of the patient's physical vitals. AGS delivers on the needs of the patient, family, and caregivers, but also provides an OEM solution for device manufacturers to offer devices that exceed all current capabilities. As we consider the art of the possible with sensor technologies, the applications are infinitely diverse among all facets of industry, commerce, and personal use.

As noted above, the systems, methods, and devices of this disclosure are configured for use with computer equipment that can control mechanical components in an electro-mechanical system, such as that shown in the figures. A computerized system of these embodiments may be used to collect raw data from an environment as described above and utilize a wide range of available analytics.

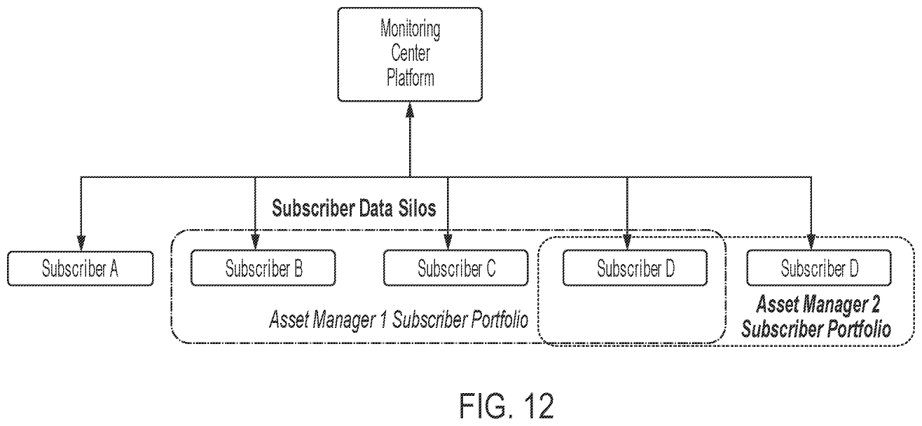

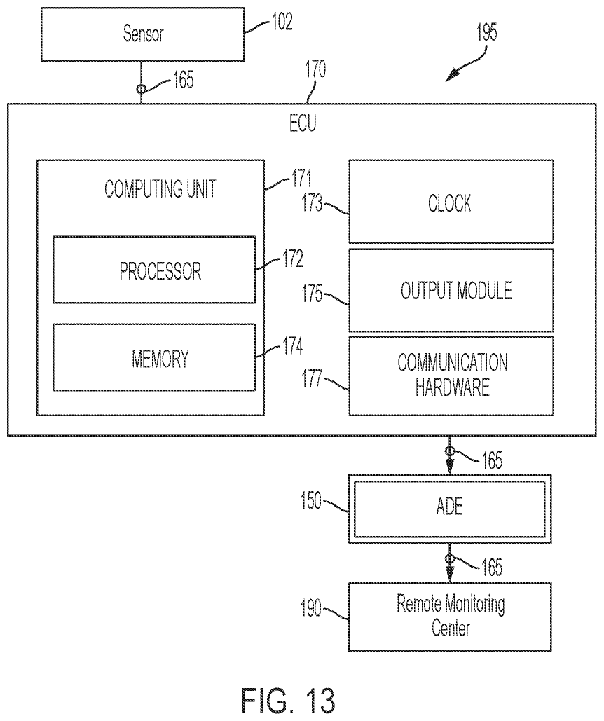

By way of example and without limiting this disclosure to any particular hardware or software, FIG. 12 illustrates a block diagram of a system herein according to one implementation. The computer processor 172 shown in FIG. 12 may comprise one or more processors, and the same or different processors may execute computer-readable instructions related to receipt of data from sensors, devices, and other computers on a bi-directional communications network 165 described herein. The system of FIG. 12 illustrates local devices (annotated as sensor 102 in the figure) connected to at least one computerized device or computer system 195 that provide the above described hub services, adaptive data exchange services 150, and remote monitoring center services 190.

The ECU, also referred to as a controller 170 which may be the hub described above, may include a computing unit 171, a system clock 173, an output module 175 and communication hardware 177. In its most basic form, the computing unit or controller 171 may include a processor 172 and a system memory 174. The processor 172 may be a standard programmable processor that performs arithmetic and logic operations necessary for operation of the controller 170. The processor 172 may be configured to execute program code encoded in tangible, computer-readable media. For example, the processor 172 may execute program code stored in the system memory 174, which may be volatile or non-volatile memory. The system memory 174 is only one example of tangible, computer-readable media. In one aspect, the computing unit 171 can be considered an integrated device such as firmware. Other examples of tangible, computer-readable media include hard drives, flash memory, or any other machine-readable storage media, wherein when the program code is loaded into and executed by a machine, such as the processor 172, the machine becomes an apparatus for practicing the disclosed subject matter.

In a system embodiment, for providing communications between local device installations 105 and a remote monitoring computer 205, the system 100 includes at least one local device 105 transmitting local device data 110 to the remote monitoring computer 205 on a bi-directional communications network 250. An interface computer 300 receives the local device data 110 and formats the local device data 110 for further transmission to the remote monitoring computer 205, wherein the interface computer 300 includes a processor 172 and computer memory 174 storing interface software that implements a method. The method includes steps of storing the local device data 110 in the computer memory 174 of the interface computer 300; transforming the local device data 110 into a formatted data set; and transmitting the formatted data set to at least one remote monitoring computer 205. The local device data may include multimedia data. The local device 105 may include a local device computer 108 implementing a local device method having the steps of receiving raw data 400 formatted according to a device manufacturer protocol and implementing local computing instructions 500 according to data protocols managed by the interface computer 300 to transform the raw data to interface protocol data. The method continues by transforming the interface protocol data into the formatted data set according to the computing instructions and an expected format protocol and transmitting the formatted data set to the remote monitoring computer.

In some embodiments, the system incorporates the local device 105 with a sensor 120 and the local device data includes at least one sensor measurement. The system may include at least one of a biometric sensor 122 and a geospatial location sensor 124. The local device 105 may include at least one in situ process sensor 128 selected from acoustic sensors, proximity sensors, tilt sensors, temperature sensors, humidity sensors, infrared imaging sensors, ultrasonic sensors, chemical sensors, biometric sensors, flow sensors, level sensors, pressure sensors, and touch sensors. The system may further incorporate a local device computer 108 connected to the local device 105, the local device computer 108 comprising may have bi-directional communications hardware 177 for receiving the local device data 110 as raw data and forwarding the raw data to a processing hub computer 350 on the network 250. The processing hub computer 350 is the local device computer 108 or a different computer on the network 250. The step of transforming the local device data 110 may include using the processing hub computer 350 to receive the local device data as raw data, parse the raw data, and transform the data per the interface computer protocol.

The method implemented by the interface computer 300 may include adaptive data exchanges 375 that collectively format the interface protocol data 380 received from the local device 105 or local device computer 108 according to at least one of the expected format protocols. The expected format protocol is stored in a database that is accessible by the interface computer 300. The method optionally includes using the processing hub computer 350 to convert the raw data from a binary format to an interface protocol format and using the interface computer to transform converted interface protocol format data to at least one of the expected format protocols for respective remote monitoring computers. In non-limiting examples, the formatted data includes JSON formatted data. In other non-limiting examples, the expected format protocols include human readable data. The human readable data is organized using JSON, FHIR, HL7, and/or XML protocols, and/or another protocol required by the remote monitoring computer. In non-limiting embodiments, the expected format protocols follow a structured template. The expected format protocols optionally allow for insertions of meta data retrieved from at least one of a database connected to the interface computer and/or the local device. In other non-limiting embodiments, the expected format protocols of the remote monitoring computers include at least a portion of the local device data in Fast Healthcare Interoperability Resources (FHIR®) format, JSON, HL7, XML protocols, and/or another protocol required by the remote monitoring computer. The system may incorporate alerting options, calendaring options, and notification options implemented by the interface computer. The alerting options and calendaring options are transmitted to the local device computer on the bi-directional communications network.

This disclosure further encompasses a computer implemented method of formatting raw data for use by a remote monitoring computer, and the computer implemented method may begin with or at least include gathering raw data from at least one local device; implementing local computing instructions according to data protocols managed by the interface computer to transform the raw data to interface protocol data; selecting a template for formatting the interface protocol data according to at least one of a plurality of expected format protocols at respective remote monitoring computers; using an adaptive data exchange software program to transform at least a portion of the interface protocol data into formatted data; and transmitting the formatted data in the expected format protocol to the remote monitoring computer. In some implementations, the raw data includes additional data that does not fit into the template for the formatted data, and the method further comprises includes transmitting the formatted data and the additional data to the remote monitoring computer. The computer implemented method may include scaling the quantity of the local device data that is formatted and transmitted to the remote monitoring computer. The method further includes scaling the quantity of alert and notification transmission by screening out extraneous transmissions based upon a set of conditions associated with the alert template. The interface computer identifies raw data for comparing to threshold data stored as an alert template at the interface computer and preparing associated alerts and/or notifications for transmission on the network. The method may also include storing the raw data as stored local device data for comparing against thresholds for how much time is allowed to elapse between readings. In optional embodiments, the method further includes gathering raw data in a fixed format and/or gathering raw data in a flexible format. The method furthermore may include providing a template at the interface computer to account for additional raw data that does not fit into the template.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the vehicle computer through any type of network, including a local area network (LAN) or a wide area network (WAN), or the connection may be made to an external computer (for example, through the Internet using an Internet Service Provider).

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

A number of implementations have been described. Nevertheless, it will be understood that various modifications may be made without departing from the spirit and scope of the claims. Accordingly, other implementations are within the scope of the following claims.

1. A system for providing communications between local device installations and a remote monitoring computer, the system comprising:
at least one local device transmitting local device data to the remote monitoring computer on a bi-directional communications network;
an interface computer receiving the local device data and formatting the local device data for further transmission to the remote monitoring computer, wherein the interface computer comprises a processor and computer memory storing interface software that implements a method comprising:
storing the local device data in the computer memory of the interface computer;
transforming the local device data into a formatted data set; and
transmitting the formatted data set to at least one remote monitoring computer.

2. The system according to claim 1, wherein the local device data comprises multimedia data.

3. The system according to claim 1, wherein the local device comprises a local device computer implementing a local device method comprising:
receiving raw data formatted according to a device manufacturer protocol;
implementing local computing instructions according to data protocols managed by the interface computer to transform the raw data to interface protocol data.

4. The system according to claim 3, further comprising:
transforming the interface protocol data into the formatted data set according to the computing instructions and an expected format protocol; and
transmitting the formatted data set to the remote monitoring computer.

5. The system according to claim 1, wherein the local device comprises a sensor and the local device data comprises at least one sensor measurement.

6. The system according to claim 5, wherein the sensor comprises at least one of a biometric sensor and a geospatial location sensor.

7. The system according to claim 1, wherein the local device comprises at least one in situ process sensor selected from the group consisting of acoustic sensors, proximity sensors, tilt sensors, temperature sensors, humidity sensors, infrared imaging sensors, ultrasonic sensors, chemical sensors, biometric sensors, flow sensors, level sensors, pressure sensors, and touch sensors.

8. The system according to claim 1, further comprising a local device computer connected to the local device, the local device computer comprising bi-directional communications hardware for receiving the local device data as raw data and forwarding the raw data to a processing hub computer on the network.

9. The system according to claim 8, wherein the processing hub computer is the local device computer or a different computer on the network.

10. The system according to claim 8, wherein transforming the local device data comprises using the processing hub computer to receive the local device data as raw data, parse the raw data, and transform the data per the interface computer protocol.

11. The system according to claim 10, wherein the method implemented by the interface computer comprises adaptive data exchanges that collectively format the interface protocol data received from the local device or local device computer according to at least one of the expected format protocols.

12. The system according to claim 11, wherein the expected format protocol is stored in a database that is accessible by the interface computer.

13. The system according to claim 12, wherein the method comprises using the processing hub computer to convert the raw data from a binary format to an interface protocol format, and using the interface computer to transform converted interface protocol format data to at least one of the expected format protocols for respective remote monitoring computers.

14. The system according to claim 13, wherein the formatted data comprises JSON formatted data.

15. The system according to claim 13, wherein the expected format protocols comprise human readable data.

16. The system according to claim 15, wherein the human readable data is organized using JSON, FHIR, HL7, and/or XML protocols, and/or another protocol required by the remote monitoring computer.

17. The system according to claim 10, wherein the expected format protocols follow a structured template.

18. The system according to claim 10, wherein the expected format protocols optionally allow for insertions of meta data retrieved from at least one of a database connected to the interface computer and/or the local device.

19. The system according to claim 10, wherein the expected format protocols of the remote monitoring computers comprise at least a portion of the local device data in Fast Healthcare Interoperability Resources (FHIR®) format, JSON, HL7, XML, protocols, and/or another protocol required by the remote monitoring computer.

20. The system according to claim 1, further comprising alerting options, calendaring options, and notification options implemented by the interface computer.

21. The system according to claim 20, wherein the alerting options and calendaring options are transmitted to the local device computer on the bi-directional communications network.

22. A computer implemented method of formatting raw data for use by a remote monitoring computer, the computer implemented method comprising:
gathering raw data from at least one local device;
implementing local computing instructions according to data protocols managed by the interface computer to transform the raw data to interface protocol data
selecting a template for formatting the interface protocol data according to at least one of a plurality of expected format protocols at respective remote monitoring computers;
using an adaptive data exchange software program to transform at least a portion of the interface protocol data into formatted data; and
transmitting the formatted data in the expected format protocol to the remote monitoring computer.

23. The computer implemented method of claim 22, wherein the raw data comprises additional data that does not fit into the template for the formatted data, and the method further comprises transmitting the formatted data and the additional data to the remote monitoring computer.

24. The computer implemented method of claim 22, wherein the method further comprises scaling the quantity of the local device data that is formatted and transmitted to the remote monitoring computer.

25. The computer implemented method of claim 24, wherein the method further comprises scaling the quantity of alert and notification transmission by screening out extraneous transmissions based upon a set of conditions associated with the alert template.

26. The computer implemented method of claim 22, further comprising using the interface computer to identify raw data for comparing to threshold data stored as an alert template at the interface computer and preparing associated alerts and/or notifications for transmission on the network.

27. The computer implemented method of claim 26, further comprising storing the raw data as stored local device data for comparing against thresholds for how much time is allowed to elapse between readings.

28. The computer implemented method of claim 22, further comprising gathering raw data in a fixed format.

29. The computer implemented method of claim 22, further comprising gathering raw data in a flexible format.

30. The computer implemented method of claim 22, further comprising providing a template at the interface computer to account for additional raw data that does not fit into the template.
